(19) United States
(12) Patent Application Publication
(10) Pub. No.: US 2008/0071085 A1
(43) Pub. Date: Mar. 20, 2008

(54) Chemical process

(75) Inventors: Mark Maybury, Macclesfield (GB); Ian Patel, Bristol (GB); Simon Nicholas George Tyler, Bristol (GB)

Correspondence Address: FISH & RICHARDSON P.C., P.O BOX 1022, MINNEAPOLIS, MN, 55440-1022, US

(73) Assignee: ASTRAZENECA AB

(21) Appl. No.: 11/897,996

(22) Filed: Aug. 31, 2007

(30) Foreign Application Priority Data
Sep. 2, 2006 (GB) 0617367.8

(51) Int. Cl. C07D 403/14

(52) U.S. Cl. 544/295

(57) ABSTRACT

A process for preparing an optically active compound of formula (II) or a salt thereof 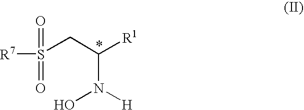
where * indicates a stereogenic centre; and R¹ and R⁷ are as defined in the specification, which process comprises the acid hydrolysis of an optically active compound of formula (IV) 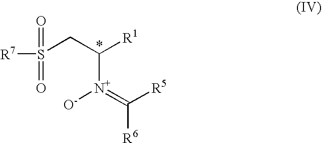
where R⁵ and R⁶ are as defined in the specification, recovering the resultant optically active compound of formula (II) as a salt, and thereafter if desired, converting the salt to a compound of formula (II). The process is suitable for the preparation of; for instance, intermediates for pharmaceutical compounds. Certain novel intermediates are also disclosed and claimed.

The present invention relates to a process for the production of certain chiral compounds, which are useful as intermediates in the preparation of pharmaceutical compounds, to certain novel compounds used in the process, as well as to methods for using these compounds in the preparation of pharmaceutical compounds.

A range of pharmaceutical compounds have been recently been published, for example in WO99/38843, WO00/75108, WO00/12478, WO 01/62742, WO03/001092, WO03/014098, WO03/014111, WO2004/006827, WO2004/006926 and WO2004/006925.

The compounds described in these references are inhibitors of one or more metalloproteinase enzymes. Metalloproteinases are a superfamily of proteinases (enzymes) whose numbers in recent years have increased dramatically. Based on structural and functional considerations these enzymes have been classified into families and subfamilies as described in N. M. Hooper (see FEBS Lett. 1994 354:1-6). Examples of metalloproteinases include the matrix metalloproteinases (MMP) such as the collagenases (MMP1, MMP8, MMP13), the gelatinases (MMP2, MMP9), the stromelysins (MMP3, MMP10, MMP11), matrilysin (MMP7), metalloelastase (MMP12), enamelysin (MMP19), the MT-MMPs (MMP14, MMP15, MMP16, MMP17); the reprolysin or adamalysin or MDC family which includes the secretases and sheddases such as TNF converting enzymes (ADAM10 and TACE); the astacin family which include enzymes such as procollagen processing proteinase (PCP); and other metalloproteinases such as aggrecanase, the endothelin converting enzyme family and the angiotensin converting enzyme family.

Metalloproteinases are believed to be important in a plethora of physiological disease processes that involve tissue remodelling such as embryonic development, bone formation and uterine remodelling during menstruation. This is based on the ability of the metalloproteinases to cleave a broad range of matrix substrates such as collagen, proteoglycan and fibronectin. Metalloproteinases are also believed to be important in the processing, or secretion, of biologically important cell mediators, such as tumour necrosis factor (TNF); and the post translational proteolysis processing, or shedding, of biologically important membrane proteins, such as the low affinity IgE receptor CD23 (for a more complete list see N. M. Hooper et al., Biochem. J. 1997 321:265-279).

Metalloproteinases have been associated with many disease conditions. Inhibition of the activity of one or more metalloproteinases may well be of benefit in these disease conditions, for example: various inflammatory and allergic diseases such as inflammation of the joint (especially rheumatoid arthritis, osteoarthritis and gout), inflammation of the gastro-intestinal tract (especially inflammatory bowel disease, ulcerative colitis and gastritis), inflammation of the skin (especially psoriasis, eczema, dermatitis); in tumour metastasis or invasion; in disease associated with uncontrolled degradation of the extracellular matrix such as osteoarthritis; in bone resorptive diseases (such as osteoporosis and Paget's disease); in diseases associated with aberrant angiogenesis; the enhanced collagen remodelling associated with diabetes, periodontal disease (such as gingivitis), corneal ulceration, ulceration of the skin, post-operative conditions (such as colonic anastomosis) and dermal wound healing; demyelinating diseases of the central and peripheral nervous systems (such as multiple sclerosis); Alzheimer's disease; and extracellular matrix remodelling observed in cardiovascular diseases such as restenosis and atherosclerosis.

A number of metalloproteinase inhibitors are known; different classes of compounds may have different degrees of potency and selectivity for inhibiting various metalloproteinases.

MMP13, or collagenase-3, was initially cloned from a cDNA library derived from a breast tumour [J. M. P. Freije et al., J. Biol. Chem. 1994 269(24):16766-16773]. PCR-RNA analysis of RNAs from a wide range of tissues indicated that MMP13 expression was limited to breast carcinomas as it was not found in breast fibroadenomas, normal or resting mammary gland, placenta, liver, ovary, uterus, prostate or parotid gland or in breast cancer cell lines (T47-D, MCF-7 and ZR75-1). Subsequent to this observation MMP13 has been detected in transformed epidermal keratinocytes [N. Johansson et al., Cell Growth Differ. 1997 8(2):243-250], squamous cell carcinomas [N. Johansson et al., Am. J. Pathol. 1997 151(2):499-508] and epidermal tumours [K. Airola et al., J. Invest. Dermatol. 1997 109(2):225-231]. These results are suggestive that MMP13 is secreted by transformed epithelial cells and may be involved in the extracellular matrix degradation and cell-matrix interaction associated with metastasis, especially as observed in invasive breast cancer lesions and in malignant epithelia growth in skin carcinogenesis.

Recent published data implies that MMP13 plays a role in the turnover of other connective tissues. For instance, consistent with MMP13's substrate specificity and preference for degrading type II collagen [P. G. Mitchell et al., J. Clin. Invest. 1996 97(3):761-768; V. Knauper et al., Biochem. J. 1996 271:1544-1550], MMP13 has been hypothesised to serve a role during primary ossification and skeletal remodelling [M. Stahle-Backdahl et al., Lab. Invest. 1997 76(5):717-728; N. Johansson et al., Dev. Dyn. 1997 208(3):387-397]; in destructive joint diseases such as rheumatoid and osteo-arthritis [D. Wernicke et al., J. Rheumatol. 1996 23:590-595; P. G. Mitchell et al., J. Clin. Invest. 1996 97(3):761-768; O. Lindy et al., Arthritis Rheum. 1997 40(8): 1391-1399]; and during the aseptic loosening of hip replacements [S. Imai et al., J. Bone Joint Surg. Br. 1998 80(4):701-710]. MMP13 has also been implicated in chronic adult periodontitis as it has been localised to the epithelium of chronically inflamed mucosa present in human gingival tissue [V. J. Uitto et al., Am. J. Pathol. 1998 152(6):1489-1499] and in remodelling of the collagenous matrix in chronic wounds [M. Vaalamo et al., J. Invest. Dermatol. 1997 109(1):96-101].

A range of compounds have therefore been developed with a view to inhibiting the metalloproteinases such as MMP-13.

Many of these include a specific functional group which can be represented as sub-formula (a) 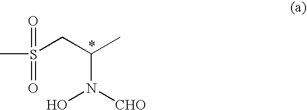
where * indicates a stereogenic centre.

This is a complex chemical moiety which poses synthetic challenges, in particular on a large scale. These challenges are exacerbated by the fact that, as with most pharmaceutical products, a single enantiomer is desirable. In particular, the stereochemical configuration which is required is represented by sub-formula (b)

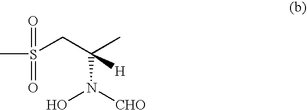

Hitherto, the routes to such compounds have generally required an optical resolution step, either of the final product, or of an intermediate utilised in the production process. Optical resolution on a large scale may be time-consuming and is inherently wasteful unless efficient re-cycling of the undesired isomer is possible.

The applicants have developed a stereoselective approach to the preparation of compounds of this type.

In particular, the invention provides a process for preparing an optically active compound of formula (II) or a salt thereof 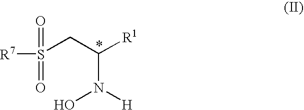
where * represents a stereogenic centre; R¹ is an optionally substituted hydrocarbyl group; R⁷ is an optionally substituted hydrocarbyl group or an optionally substituted heterocyclic group, which process comprises the acid hydrolysis of an optically active compound of formula (IV) 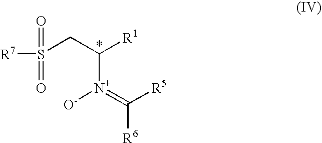
where R¹ and R⁷ are as defined above; and one of R⁵ or R⁶ is an optionally substituted aromatic group or electron-withdrawing group, and the other is an optionally substituted alkyl group, recovering the resultant optically active salt, and thereafter if desired, converting the salt to a compound of formula (II).

Suitable optionally substituted aromatic groups R⁵ or R⁶ are aryl or heteroaryl groups as defined below. In addition, however, R⁵ or R⁶ may be a different electron-withdrawing group, such as an electron-withdrawing functional group or a hydrocarbyl group substituted with a functional group to make it electron-withdrawing in character. Particular examples of such electron-withdrawing groups include cyano, carboxy, carboalkoxy, carbamoyl, acyl, nitro and perfluoroalkyl.

As used herein, the expression “optically active” refers to compounds which have a preponderance (greater than 50%) and preferably a significant preponderance such as in excess of 80% of a single enantiomeric form, giving rise to optical activity.

Hydrolysis of the compound of formula (IV) may be carried out such that the chiral integrity of the starting material is largely retained. Thus, where the compound of formula (IV) contains a preponderance of a particular enantiomer, such as a compound of formula (IVA), 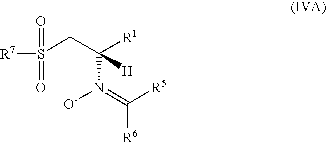
hydrolysis leads to an optically active product, containing a preponderance of a particular enantiomer of the compound of formula (II). Thus for instance, where the starting material is a compound of formula (IVA), where R⁵ is the optionally substituted aromatic group or electron-withdrawing group, and R⁶ is the optionally substituted alkyl group such as methyl, the product of formula (II) will contain a preponderance of the enantiomer of formula (IIA) 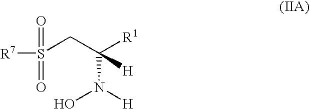
where R¹ and R⁷ are as defined above.

Suitably the reaction is carried out in an organic solvent such as acetonitrile, toluene, tetrahydrofuran (THF), ethyl acetate, butyl acetate or mixtures thereof. These may be combined with anti-solvents such as isopropyl acetate, anisole, butyl acetate, tert-butyl methyl ether (MTBE) as necessary or desired in order to ensure that the desired product is obtained effectively by crystallisation.

The reaction is suitably carried out at moderate temperatures, for example of from 0 to 50° C. and conveniently at about 20° C.

The acid used in the hydrolysis reaction may be any organic acid, and examples include trifluoroacetic acid, trichloroacetic acid, p-toluenesulfonic acid monohydrate, oxalic acid, oxalic acid dihydrate, dichloroacetic acid, 2,4-dinitrobenzoic acid, 2,4,6-trihydroxybenzoic acid monohydrate, maleic acid, 2-nitrobenzoic acid, pyruvic acid, 2-ketoglutaric acid, 2-oxobutanoic acid, oxalacetic acid, 3,5-dinitrobenzoic acid, malonic acid, chloroacetic acid, fumaric acid, 2,4-dihydroxybenzoic acid, citric acid, glyoxylic acid monohydrate, 4-nitrobenzoic acid, 3-nitrobenzoic acid, 4-fluorobenzoic acid, formic acid, benzoic acid, succinic acid, glutaric acid, acetic acid and propanoic acid.

In particular, however, the acid used is an enantiomerically pure chiral acid, as this enables the differential crystallisation of the diastereomeric salts. Suitable chiral acids include (+)-camphor-10-sulfonic acid, (−)-camphor-10-sulfonic acid, 2,3;4,6-di-O-isopropylidene-2-keto-L-gulonic acid, dibenzoyl-L-tartaric acid, dibenzoyl-D-tartaric acid, L-tartaric acid, D-tartaric acid, L-malic acid, D-malic acid and (+)-camphoric acid.

The selection of acid utilised for optimum product recovery will vary depending upon factors such as the precise nature of the compound of formula (II). However, it will generally be preferable to utilise an acid which gives a crystalline salt of the compound of formula (II).

The applicants have found that L-tartaric acid can be a preferred acid for use in the process in some instances.

Compounds of formula (IV) are suitably prepared by reacting an optically active compound of formula (V) 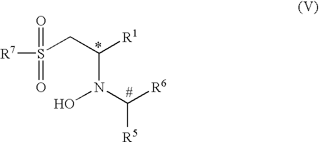
where *, R¹, R⁷, R⁸ and R⁶ are as defined above, and # represents a second stereogenic centre, with an oxidising agent. Suitably, where R⁵ is the aromatic group or electron-withdrawing group, such as cyano, and R⁶ is alkyl such as methyl, the compound of formula (V) is, or comprises a preponderance of the diastereomer of formula (VA) 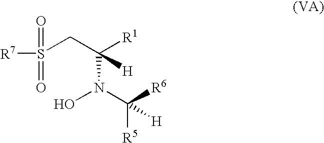
where R¹, R⁷, R⁸ and R⁶ are as defined above.

Suitable oxidising agents include hydrogen peroxide, m-chloroperbenzoic acid, a halosuccinimide, such as N-bromosuccinimide or N-chlorosuccinimide, 1,3-dibromo-5,5-dimethylhydantoin, trichloroisocyanuric acid, Oxone®, an alkali metal permanganate such as potassium permanganate or an alkali metal hypochlorite, such as sodium hypochlorite.

In particular however, the oxidising agent used is an alkali metal hypochlorite, such as sodium hypochlorite. Suitably the sodium hypochlorite used has an available chlorine content of less than 15%, and preferably of about 5% (ca. 0.75 M) which provides for better stability on storage.

The reaction is suitably effected in an organic solvent such as tetrahydrofuran (THF), toluene, benzonitrile, acetonitrile or mixtures thereof. Toluene is a preferred solvent in some cases, mediating very clean reaction, although tetrahydrofuran (THF) either alone or in mixture with a nitrile may deliver a more rapid reaction rate.

If required, a phase transfer catalyst such as tetrabutyl-, tetraoctyl-, or tetrahexadecylammonium bromide may be included in the reaction to enhance the reaction rate.

In a particular preferred embodiment, the compound of formula (IV) produced is converted to a compound of formula (II) in situ, without first being isolated. This will enhance both the manufacturability and economics of the process.

The compound of formula (V) is suitably prepared by reacting a compound of formula (VI) 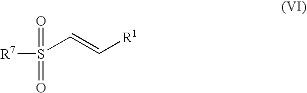
wherein R¹ and R⁷ are as defined above, with an optically active compound of formula (VII) or a salt thereof 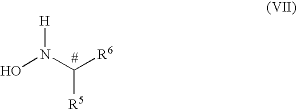
wherein R⁵ and R⁶ are as defined above, and # indicates a stereogenic centre, in the presence of a base.

This reaction, a reverse Cope conjugate addition, is stereoselective. Thus where the compound of formula (VII) is a compound of formula (VIIA), 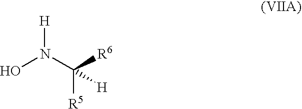
where R⁵ is an aromatic group such as phenyl, p-methoxyphenyl or naphthyl or an electron-withdrawing group such as cyano, and R⁶ is an alkyl group such as methyl, reaction with a compound of formula (VI) leads to a product which is optically active, having a preponderance of the diastereomer of formula (VA). The selective introduction of the stereogenic centre β to the sulfonamide group is very useful in the production process insofar as it eliminates the need for later resolution steps, provided the chiral integrity is retained during the subsequent steps, which the production of the intermediate nitrone of formula (IV) allows, as outlined above.

The reaction between the compounds of formula (VI) and (VII) is suitably carried out in a solvent such as water, an alkanol, for instance methanol, ethanol or isopropanol, 2-methoxyethanol, tetrahydrofuran (THF), industrial methylated spirits (IMS), alkyl ethers such as diethyl ether, dibutyl ether, diisopropyl ether, tert-butyl methyl ether (MTBE) or di(ethylene glycol), alkyl acetates such as ethyl acetate, butyl acetate, tert-butyl acetate or isopropyl acetate, alkyl ketones such as acetone, nitriles such as acetonitrile or benzonitrile, N,N-dimethylformamide (DMF), dimethyl sulfoxide (DMSO), toluene, halocarbons such as dichloromethane (DCM), 1,2-dichloroethane (DCE) and chlorobenzene, N-methylpyrrolidone (NMP), N,N-dimethylacetamide (DMA) or mixtures thereof.

Preferred solvents include ethanol, industrial methylated spirits (IMS), tetrahydrofuran (THF), ethyl acetate and tert-butyl acetate.

The reaction is carried out in the presence of a base, and suitable bases include pyridine, alkali metal hydroxides, carbonates or bicarbonates such as sodium hydroxide, potassium carbonate or sodium bicarbonate, amines such as 4-(dimethylamino)pyridine (DMAP), 4-aminopyridine, benzylamine, triethylamine, piperazine, 1,4-diazabicyclo[2,2,2]octane (DABCO™), morpholine, N,N,N′,N′-tetramethethylenediamine (TMEDA), 1,8-diazabicyclo[5.4.0]undec-7-ene (DBU) or quinuclindine.

Particular bases are triethylamine and DABCO™. DABCO™ is a particularly preferred base.

The reaction is suitably carried out at moderate temperatures, for example of from 0 to 50° C. and conveniently at about 20° C.

In particular R⁷ is a group NR²R³, where R² and R³ are as defined hereinafter, making the compound of formula (VI) a vinyl sulfonamide. The applicants have found that a vinyl sulfonamide of formula (VI) can act as the electrophilic partner in a reverse-Cope addition of this type, in spite of it being a relatively poor Michael acceptor. However the applicants have found that this reaction can proceed in good yields and with good stereoselectivity, depending upon the choice of appropriate bases and solvents such as those listed above.

Compounds of formula (VII) are known compounds (see for example H. Tokuamaya et al., Synthesis 2000 9:1299-1304) and can be made using conventional methods. A preferred method for preparing compounds of formula (VII) is described and claimed in a co-pending application of the applicants of even date.

Compounds of formula (VI) may be prepared by conventional chemical methods, and the precise route will depend upon the particular values of R¹ and R⁷ present in the molecule.

For example, compounds of formula (VI) may be prepared by formal elimination of water from a compound of formula (VIII) 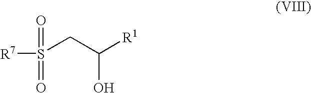
where R¹ and R⁷ are as defined above.

Reaction is suitably carried out in an organic solvent such as dichloromethane (DCM), using a reagent such as methanesulfonyl chloride, in the presence of a base such as triethylamine. Temperatures in the range of from −5 to 25° C. are suitably employed.

Compounds of formula (VIII) in their turn may be prepared by reduction of a compound of formula (IX) 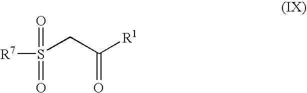
wherein R¹ and R⁷ are as defined above.

Suitable reducing agents in this case include alkali metal borohydrides such as sodium borohydride. The reaction is suitably carried out in an organic solvent system such as aqueous tetrahydrofuran (THF) or methanol/dichloromethane (DCM) at temperatures in the range of from 20 to 50° C.

Compounds of formula (IX) are suitably prepared by reacting a compound of formula (X) 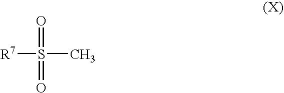
wherein R⁷ is as defined above, with a compound of formula (XI) 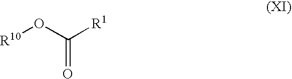
wherein R¹ is as defined above and R¹⁰ is an alkyl group, such as C₁₋₆ alkyl like ethyl.

The condensation of these two compounds is suitably effected using lithium hexamethyldisilazide (LiHMDS) in an organic solvent such as tetrahydrofuran at temperatures of from −78 to −10° C.

Compounds of formula (X) and (XI) are known compounds or they can be prepared from known compounds by methods which would be readily apparent to a skilled person. For example, certain compounds of formula (XI) and preparation methods therefore are described in WO2004/006927.

Compounds of formula (X) will be prepared using various methods depending upon the particular nature of the R⁷ group. Particular examples of compounds of formula (X) and their preparation are described in WO01/62742.

Once obtained using the method of the invention, optically active compounds of formula (II) or salts thereof are suitably converted to a compound of formula (I) or a salt thereof 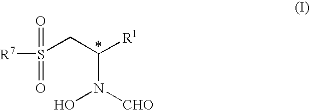
where R¹ and R⁷ are as defined above and * indicates a stereogenic centre; by reaction with a compound of formula (III) 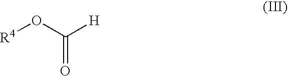
where R⁴ is an alkyl, aralkyl, aryl or acyl group, any of which may be optionally substituted, for example with a functional group such as halo and in particular fluoro. Particular examples of groups R⁴ include acetyl, ethyl or 2,2,2-trifluoroethyl. This reaction is suitably carried out at temperatures in the range of from
−10 to 60° C., preferably at about 0° C. A suitable solvent for the reaction is formic acid which, when combined with an anhydride such as acetic anhydride, gives rise to a compound of formula (III), and particularly a compound of formula (IIIA), in situ.

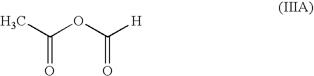

In particular, the compound of formula (II) is a compound of formula (IIA) as defined above, and so the compound of formula (I) obtained is a compound of formula (IA) or a salt thereof 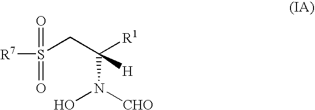
where R¹ and R⁷ are as defined above in relation to formula (II).

Certain of these compound s may be used as metalloproteinase inhibitors, as illustrated, for example in WO99/38843, WO00/75108, WO00/12478, WO01/062742, WO03/001092, WO03/014098, WO03/0141111, WO2004/006827, WO2004/006926 and WO2004/006925.

As used herein the term “hydrocarbyl” refers to alkyl, alkenyl, alkynyl, cycloalkyl, aryl, or aralkyl groups.

As used herein the expression “alkyl” includes groups having up to 10, preferably up to 6 carbon atoms, which may be both straight-chain and branched-chain alkyl groups such as propyl, isopropyl and tert-butyl. Similarly the terms “alkenyl” and “alkynyl” include unsaturated groups having from 2 to 10 and preferably from 2 to 6 carbon atoms, which may also be straight-chain or branched-chain. The term “cycloalkyl” includes C₃₋₈ cycloalkyl groups such as cyclopropyl, cyclobutyl, cyclopentyl, cyclohexyl and cycloheptyl.

An analogous convention applies to other generic terms, for example “alkoxy” includes alkyl groups as defined above which are linked by way of an oxygen and so includes methoxy, ethoxy, propoxy, etc.

The term “aryl” refers to aromatic hydrocarbon rings such as phenyl or naphthyl. The terms “heterocyclic” or “heterocyclyl” include ring structures that may be mono- or bicyclic and contain from 3 to 15 atoms, at least one of which, and suitably from 1 to 4 of which, is a heteroatom such as oxygen, sulfur or nitrogen. Rings may be aromatic, non-aromatic or partially aromatic in the sense that one ring of a fused ring system may be aromatic and the other non-aromatic. Particular examples of such ring systems include furyl, benzofuranyl, tetrahydrofuryl, chromanyl, thienyl, benzothienyl, pyridyl, piperidinyl, quinolyl, 1,2,3,4-tetrahydroquinolinyl, isoquinolyl, 1,2,3,4-tetrahydroisoquinolinyl, pyrazinyl, piperazinyl, pyrimidinyl, pyridazinyl, quinoxalinyl, quinazolinyl, cinnolinyl, pyrrolyl, pyrrolidinyl, indolyl, indolinyl, imidazolyl, benzimidazolyl, pyrazolyl, indazolyl, oxazolyl, benzoxazolyl, isoxazolyl, thiazolyl, benzothiazolyl, isothiazolyl, morpholinyl, 4H-1,4-benzoxazinyl, 4H-1,4-benzothiazinyl, 1,2,3-triazolyl, 1,2,4-triazolyl, oxadiazolyl, furazanyl, thiadiazolyl, tetrazolyl, dibenzofuranyl, dibenzothienyl oxiranyl, oxetanyl, azetidinyl, tetrahydropyranyl, oxepanyl, oxazepanyl, tetrahydro-1,4-thiazinyl, 1,1-dioxotetrahydro-1,4-thiazinyl, homopiperidinyl, homopiperazinyl, dihydropyridinyl, tetrahydropyridinyl, dihydropyrimidinyl, tetrahydropyrimidinyl, tetrahydrothienyl, tetrahydrothiopyranyl or thiomorpholinyl.

Where rings include nitrogen atoms, these may carry a hydrogen atom or a substituent group such as a C₁₋₆ alkyl group if required to fulfil the bonding requirements of nitrogen, or they may be linked to the rest of the structure by way of the nitrogen atom. A nitrogen atom within a heterocyclyl group may be oxidised to give the corresponding N-oxide.

The term “heteroaryl” refers specifically to heterocyclic rings which are aromatic in nature such as pyridyl, pyrimidinyl, etc. The term “aralkyl” refers to alkyl groups which are substituted by aryl groups, and a particular example is benzyl. Examples of saturated heterocyclic groups include morpholine or tetrahydropyranyl.

The term “halo” or “halogen” includes fluorine, chlorine, bromine and iodine.

Suitable optional substituents for hydrocarbyl groups R¹ and hydrocarbyl or heterocyclic groups R⁷ include functional groups, or aryl or heterocyclic groups either of which may be optionally substituted with functional groups.

Examples of functional groups include halo, nitro, cyano, NR¹¹R¹², OR¹³, C(O)ₙR¹³, C(O)NR¹¹R¹²⁻, OC(O)NR¹¹R¹²⁻, NR¹³C(O)NR¹⁴, NR¹³C(O)NR¹¹R¹², —N═CR¹³R¹⁴, S(O)ₘR¹³, S(O)ₘNR¹¹R¹² or NR¹³S(O)ₙR¹⁴ where R¹¹, R¹², R¹³ and R¹⁴ are independently selected from hydrogen, optionally substituted heterocyclyl, optionally substituted hydrocarbyl, or R¹¹ and R¹² together with the atom to which they are attached, form an optionally substituted heterocyclyl ring as defined above which optionally contains further heteroatoms such as S(O)ₙ, oxygen and nitrogen, where n is an integer of 1 or 2, m is 0 or an integer of 1 to 3.

Any cycloalkyl, aryl or heterocyclic groups R¹ and R⁷ may also be substituted by alkyl, alkenyl or alkynyl groups, which may themselves be optionally substituted by a functional group, an aryl group or a heterocyclic group as described above.

Suitable optional substituents for hydrocarbyl or heterocyclyl groups R¹¹, R¹², R¹³ and R¹⁴ include halo, perhaloalkyl such as trifluoromethyl, mercapto, hydroxy, carboxy, alkoxy, heteroaryl, heteroaryloxy, alkenyloxy, alkynyloxy, alkoxyalkoxy, aryloxy (where the aryl group may be substituted by halo, nitro, or hydroxy), cyano, nitro, amino, mono- or di-alkyl amino, alkylthio, alkylsulfinyl, alkylsulfonyl or oximino.

Where R¹¹ and R¹² together form a heterocyclic group, this may be optionally substituted by hydrocarbyl such as alkyl as well as those substituents listed above for hydrocarbyl groups R¹¹, R¹², R¹³ and R¹⁴.

Suitable groups R¹ include optionally substituted C₁₋₆ alkyl, C₂₋₆ alkenyl, aryl, aryl-C₁₋₆ alkyl, heteroaryl, saturated heterocyclyl and saturated heterocyclylalkyl, any of which may be optionally substituted as described above.

In particular R¹ is selected from C₁₋₆ alkyl, C₅₋₇ cycloalkyl, up to C₁₀ aryl, up to C₁₀ heteroaryl, up to C₁₋₂ aralkyl, or up to C₁₋₂ heteroarylalkyl, all of which may be optionally substituted by up to three groups independently selected from NO₂, CF₃, halogen, C₁₋₄ alkyl, carboxy-C₁₋₄ alkyl, up to C₆ cycloalkyl, OR₈, SR₈, C₁₋₄ alkyl substituted with OR⁸, SR₈ (and its oxidised analogues), NR₈, N—Y—R₈, or C₁₋₄ alkyl-Y—N₈,

R₈ is hydrogen, C₁₋₆ alkyl, up to C₁₀ aryl or up to C₁₀ heteroaryl or up to C₉ aralkyl, each independently optionally substituted by halogen, NO₂, CN, CF₃, C₁₋₆ alkyl, SC₁₋₆ alkyl, SOC₁₋₆ alkyl, SO₂C₁₋₆ alkyl or C₁₋₆ alkoxy, and Y is selected from —SO₂— and —CO—.

In a particular embodiment, R¹ represents an optionally substituted group selected from C₁₋₆ alkyl, C₅₋₇ cycloalkyl, a saturated heterocyclyl, aryl, heteroaryl, aryl-C₁₋₆ alkyl, heteroaryl-C₁₋₆ alkyl, cycloalkyl-C₁₋₆ alkyl or saturated heterocyclyl-C₁₋₆ alkyl.

Suitable optional substitutents for R¹ are as listed above. However preferably, when R¹ is substituted, this is preferably by one or two substituents, which may be the same or different, selected from C₁₋₄ alkyl, halogen, CF₃ and CN.

A preferred substituent is halogen, particularly fluorine.

Preferably where R¹ is substituted, it is monosubstituted.

More particularly, R¹ is selected 3-chlorophenyl, 4-chlorophenyl, 3-pyridyl, 2-pyridylpropyl, 2- or 4-pyrimidinylethyl (optionally monosubstituted by fluorine), 2- or 4-pyrimidinylpropyl, 2-(2-pyrimidinyl)propyl (optionally monosubstitued by fluorine); especially 2-pyrimidinylpropyl, 2-(2-pyrimidinyl)propyl (optionally monosubstitued by fluorine) or 5-fluoro-2-pyrimidinylethyl.

Examples of the group R⁷ include those groups listed as “B” in WO99/38843 which are C₁₋₆ alkyl-aryl, C₁₋₆ alkyl, cycloalkyl, C₁₋₆ alkyl-cycloalkyl, cycloalkenyl, heterocycloalkenyl, C₁₋₆ alkyl-heteroaryl, saturated heterocyclyl (heterocycloalkyl), C₁₋₆ alkyl-heterocycloalkyl, aryl, and heteroaryl, any of which groups can optionally be substituted by a substituent selected from the group consisting of R¹⁵, C₁₋₆ alkyl-R¹⁵, C₂₋₄ alkenyl-R⁶⁵, aryl (optionally substituted with R¹⁵), aryl-C₁₋₆ alkyl-R¹⁵, C₁₋₆ alkyl-aryl (optionally substituted with R¹⁵), C₁₋₆ alkyl-heteroaryl (optionally substituted with R¹⁵), aryl-C₂₋₆ alkenyl-R¹⁶, heteroaryl (optionally substituted with R¹⁵), heteroaryl-C₁₋₆ alkyl-R¹⁵, cycloalkyl (optionally substituted with R¹⁵), and heterocycloalkyl (optionally substituted with R¹⁵), where R¹⁵ is selected from the group consisting of C₁₋₆ alkyl, halogen, CN, NO₂, N(R¹⁷)₂, OR¹⁷, COR¹⁷, C(═NOR¹⁸)R¹⁷, CO₂R¹⁹, CON(R⁷)₂, NR⁶R⁷, S(O)₀₋₂R¹⁸, and SO₂N(R¹⁵)₂; where R¹⁵ is H or a substituent selected from the group consisting of C₁₋₆ alkyl, aryl, aryl-C₁₋₆ alkyl, heteroaryl, heteroaryl-C₁₋₆ alkyl, cycloalkyl, cycloalkyl-C₁₋₆ alkyl, saturated heterocyclyl, and saturated heterocycloalkyl-C₁₋₆ alkyl, wherein said substituent is optionally substituted with R¹⁸, COR¹⁸, SO₀₋₂R¹⁸, CO₂R¹⁸, OR¹⁸, CONR¹⁹R¹⁸, NR¹⁹R¹⁸, halogen, CN, SO₂NR¹⁹R¹⁸, or NO₂, and for each case of N(R¹⁵)₂, the R¹⁵ groups are the same or different, or N(R¹⁵)₂ is saturated heterocyclyl optionally substituted with R¹⁸, COR¹⁸, SO₀₋₂R¹⁸, CO₂R¹⁸, OR¹⁸, CONR¹⁹R¹⁸, NR¹⁹R¹⁸, NR¹⁹R¹⁸, halogen, CN, SO₂NR¹⁹R¹⁸, or NO₂;

R¹⁶ is selected from the group consisting of COR¹⁵CON(R¹⁵)₂, CO₂R¹⁸, and SO₂R¹⁸;

R¹⁸ is selected from the group consisting of C₁₋₆ alkyl, aryl, aryl-C₁₋₆ alkyl, heteroaryl, and heteroaryl-C₁₋₆ alkyl;

R¹⁹ is hydrogen or C₁₋₆ alkyl.

In particular R⁷ is a substituted saturated heterocyclic group. More specifically, R⁷ is a group NR²R³ where R² and R³, together with the nitrogen atom to which they are attached form an optionally substituted saturated ring, which optionally contains further heteroatoms.

Specific examples of such groups are represented as groups of sub-formula (c) 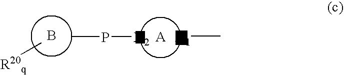
where X₁ and X₂ are independently selected from N and C;
ring B is a monocyclic or bicyclic cycloalkyl, aryl or heteroaryl ring comprising up to 12 ring atoms and containing one or more heteroatoms independently chosen from N, O, and S;
or ring B may be biphenyl;
or ring B may be linked to ring A by a C₁₋₄ alkyl or a C₁₋₄ alkoxy chain linking the 2-position of ring B with a carbon atom α to X²;
q is 0, 1, 2 or 3 and each R²⁰ is independently selected from halogen, NO₂, COOR or a group OR²³ wherein R is hydrogen or C₁₋₆ alkyl, CN, CF₃, C₁₋₆ alkyl, SC₁₋₆ alkyl, SOC₁₋₆ alkyl, SO₂C₁₋₆ alkyl, C₁₋₆ alkoxy and up to C₁₀ aryloxy and R²³ represents a group selected from C₁₋₆ alkyl or aryl, which said group is substituted by one or more fluorine groups;
P is —(CH₂)ₛ— wherein s is 0, 1 or 2, or P is an alkene or alkyne chain of up to six carbon atoms; and where X₂ is C, P may be a group -Z-, —(CH[R²²])ₜ-Z-, -Z-(CH[R²²]ₜ— or -Z-(CH[R²²])_-Z-, wherein Z is selected from —CO—, —S—, SO—, —SO₂—, —NR²²—, or —O— wherein t is 1 or 2, or P may be selected from —CO—N(R²²)—, —N(R²²)—CO—, —SO₂N(R²²)— and —N(R²²)SO₂—, and R²² is hydrogen, C₁₋₆ alkyl, up to C₁₀ aralkyl or up to C₉ heteroaryl; and
ring A is a 5 to 7 membered saturated ring which is optionally mono- or di-substituted by groups independently selected from halogen, C₁₋₆ alkyl, C₁₋₆ alkoxy or an oxo group wherein the C₁₋₆ alkyl groups may be optionally substituted by halo.

Suitably where ring A has an oxo substituent, it is adjacent to a ring nitrogen atom.

Preferably X₁ and X₂ are both N. Preferably ring Z is unsubstituted.

Suitably ring B is a monocyclic or bicyclic aryl or heteroaryl ring having up to 10 ring atoms, especially a monocyclic aryl or heteroaryl having up to 7 ring atoms, more especially monocyclic aryl or heteroaryl having up to 6 ring atoms, such as a phenyl or pyridyl ring.

Suitably P is —(CH₂)₈— wherein s is 0 or 1, or P is —O—, or —CO—N(R²²)—.

Most preferably s is 0.

Particular examples of R²⁰ include halogen such as chlorine, bromine or fluorine, NO₂, CF₃, methyl, ethyl, methoxy or ethoxy, and particularly, methoxy or fluorine. Alternatively R²⁰ is CF₃. Suitably q is 1.

Alternatively R²⁰ is a group selected from C₁₋₆ alkyl or aryl, which said group is substituted by one or more fluorine groups. Particular examples of such groups are a C₁₋₆ alkyl group substituted by one to five fluorine groups, for instance, CF₂CHF₂ or CH₂CF₃.

Another particular examples of possible groups R⁷ include the following sub-formula (d) 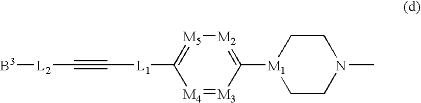
where B³ is selected from hydrogen, C₁₋₆ alkyl, C₃₋₁₂ cycloalkyl, up to C₁₋₂ aryl, and up to C₁₋₂ heteroaryl, any of the alkyl, cycloalkyl, aryl or heteraryl groups being optionally substituted by up to 3 groups selected from OH, NO₂, CF₃, CN, halogen, SC₁₋₄ alkyl, SOC₁₋₄ alkyl, SO₂C₁₋₄ alkyl, C₁₋₄ alkyl, C₁₋₄ alkoxy;
L₁ and L₂ are independently selected from direct bonds and C₁₋₆ alkyl, and
M₁, M₂, M₃, M₄ and M₅ are each independently selected from N and C.

Examples of preferred groups within sub-formula (d) are as set out in WO03/014111.

Particular examples of compounds of the formula (IA) are compounds of formula (X) 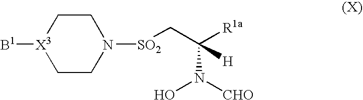
wherein B′ represents a phenyl group monosubstituted at the 3- or 4-position by halogen or trifluoromethyl, or disubstituted at the 3- and 4-positions by halogen (which may be the same or different); or B represents a 2-pyridyl or 2-pyridyloxy group monosubstituted at the 4-, 5- or 6-position by halogen, trifluoromethyl, cyano or C₁₋₄ alkyl; or B represents a 4-pyrimidinyl group optionally substituted at the 6-position by halogen or C₁₋₄ alkyl;
X³ represents a carbon or nitrogen atom;
R^{1a} represents a trimethyl-1-hydantoin C₂₋₄ alkyl or a trimethyl-3-hydantoin C₂₋₄ alkyl group; phenyl or C₂₋₄ alkylphenyl monosubstituted at the 3- or 4-position by halogen, trifluoromethyl, thio or C₁₋₃ alkyl or C₁₋₃ alkoxy; phenyl-SO₂NHC₂₋₄ alkyl; 2-pyridyl or 2-pyridyl C₂₋₄ alkyl; 3-pyridyl or 3-pyridyl C₂₋₄ alkyl; 2-pyrimidine-SCH₂CH₂; 2- or 4-pyrimidinyl C₂₋₄ alkyl optionally monosubstituted by one of halogen, trifluoromethyl, C₁₋₃ alkyl, C₁₋₃ alkyloxy, 2-pyrazinyl optionally substituted by halogen or 2-pyrazinyl C₂₋₄ alkyl optionally substituted by halogen.

In particular, B′ represents 4-chlorophenyl, 4-fluorophenyl, 4-bromophenyl or 4-trifluorophenyl; 2-pyridyl or 2-pyridyloxy monosubstituted at the 4- or 5-position such as 5-chloro-2-pyridyl, 5-bromo-2-pyridyl, 5-fluoro-2-pyridyl, 5-trifluoromethyl-2-pyridyl, 5-cyano-2-pyridyl, 5-methyl-2-pyridyl; especially 4-fluorophenyl, 5-chloro-2-pyridyl or 5-trifluoromethyl-2-pyridyl;

X³ represents a nitrogen atom;

R^{1a} is 3-chlorophenyl, 4-chlorophenyl, 3-pyridyl, 2-pyridylpropyl, 2- or 4-pyrimidinylethyl (optionally monosubstituted by fluorine), 2- or 4-pyrimidinylpropyl, 2-(2-pyrimidinyl)propyl (optionally monosubstitued by fluorine); especially 2-pyrimidinylpropyl, 2-(2-pyrimidinyl)propyl (optionally monosubstitued by fluorine) or 5-fluoro-2-pyrimidinylethyl.

In an alternative embodiment, the compound of formula (IA) is a compound of formula (XI) or a pharmaceutically acceptable salt, prodrug or solvate thereof 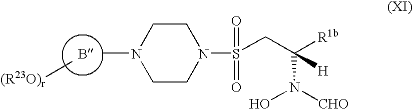
wherein ring B″ represents a monocyclic aryl ring having six ring atoms or a monocyclic heteroaryl ring having up to six ring atoms and containing one or more ring heteroatoms wherein each said heteroatom is nitrogen;
R²³ is as Defined Above;
r is 1, 2 or 3; and
R^{1b} represents an optionally substituted group selected from C₁₋₆ alkyl, C₅₋₇ cycloalkyl, a saturated heterocyclyl, aryl, heteroaryl, aryl C₁₋₆ alkyl, heteroaryl-C₁₋₆ alkyl, cycloalkyl-C₁₋₆ alkyl or saturated heterocyclyl-C₁₋₆ alkyl.

The term “prodrug” as used herein refers to derivatives of the compounds which are hydrolysed in vivo to form compounds of formula (I). These may include esters and amide derivatives in particular pharmaceutically acceptable ester and pharmaceutically acceptable amide derivatives, such as alkyl esters or alkyl amides. They may be prepared by conventional methods.

It is also to be understood that certain compounds can exist in solvated as well as unsolvated forms such as, for example, hydrated forms. A solvated form is referred to herein as a “solvate”.

Particular examples of R^{1b} will be those groups listed above for R¹ which fall within the definition of R^{1b}. Particular examples of R¹ are tetrahydropyranyl, 2-pyrimidinyl-CH₂CH₂—, 2-pyrimidinyl-CH₂CH₂CH₂— or 5-F-2-pyrimidinyl-CH₂CH₂—.

Suitably r is 1 and preferred R²³ groups are as defined above.

Suitable groups B″ are phenyl, pyridinyl or pyrimidinyl.

In the process described above, one of R⁵ or R⁶ is an optionally substituted aromatic group or an electron-withdrawing group such as cyano and the other is an optionally substituted alkyl group. Aromatic groups include aryl or heteroaryl groups as defined above.

Suitable optional substitutents for R⁵ or R⁶ include functional groups as defined above. Additional possible substituents for the alkyl group R⁵ or R⁶ are aryl, cycloalkyl or heterocyclic groups, whilst aromatic groups R⁵ or R⁶ may additionally be substituted with alkyl groups which may be optionally substituted with functional groups.

In particular, R⁵ or R⁶ may be substituted with a group OR³, such as hydroxy.

Preferably, R⁵ or R⁶ are unsubstituted.

In particular, one of R⁵ or R⁶ is C₁₋₃ alkyl such as methyl, and the other is either phenyl, p-methoxyphenyl, pyridyl or napthyl, and preferably phenyl.

Certain intermediates described above are novel compounds and these form part of the invention. In particular, compounds of formula (IV), (IVA), (V) and (VA) where R⁷ is a group NR²R³ as defined above are novel and form a further aspect of the invention, as do salts of compounds of formula (II) and (IIA) with optically active acids.

The invention will now be particularly described by way of example.

### EXAMPLE 1

### Preparation of Compound J

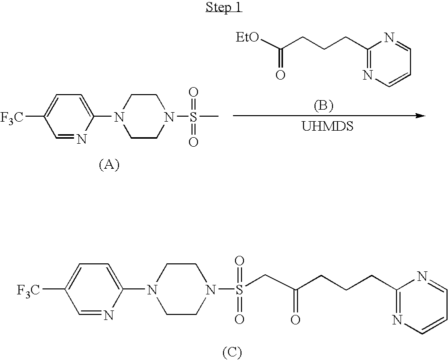

To a suspension of Compound A (10.0 g) in tetrahydrofuran (150 mL) under nitrogen was charged lithium hexamethyldisilazide (1 M in tetrahydrofuran, 61.4 mL) at −15° C. over 60 min. After holding for 90 min, Compound B (8.2 g) was charged over 75 min, whilst maintaining the temperature at −15° C. After a hold of 30 min at −15° C. the reaction was quenched with acetic acid (80% w/w, 18.1 mL). The mixture was warmed to 40° C. before dilution with water (50 mL). The aqueous phase is removed and the organic phase was distilled, replacing the distillates with butyl acetate (100 mL). The batch temperature was adjusted to 80° C. and then cooled to 10° C. at a rate of 20° C. per hour. After holding for 2 h at 10° C., the product (Compound C) was isolated by filtration and dried under reduced pressure at up to 50° C. (12.3 g, 78%).

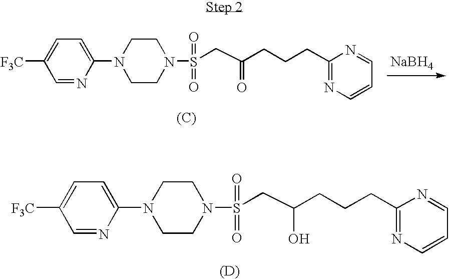

To Compound C (20.0 g) was added tetrahydrofuran (120 mL) and water (120 mL) and the reaction mixture warmed to 35° C. under nitrogen. Sodium borohydride (1.0 g) in aq. sodium hydroxide (2 M, 10.0 mL) was charged over 2 h. After a hold of 4 h at 35° C. the reaction was quenched by the addition of aq. hydrochloric acid (5 M, 32.8 mL) over 1 h. The batch was filtered and the filtrate warmed to 35° C. before aq. sodium hydroxide (8 M, 14.0 mL) was added over 1 h. Further aq. sodium hydroxide (2 M, ca. 5 mL) was added as necessary to adjust the batch to pH 7. The batch temperature was cooled to 0° C. over 2 h. After holding for 1 h at 0° C., the product (Compound D) was isolated by filtration, washed with water (2×20 mL) and dried under reduced pressure at 43° C. (19.4 g, 96%).

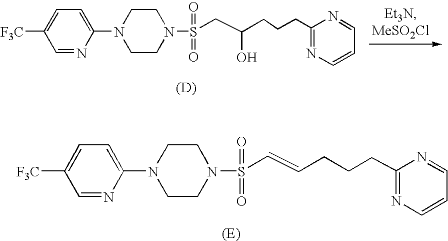

To Compound D (15.0 g) was added dichloromethane (450 mL) and the reaction mixture warmed to 30° C. under nitrogen. The slurry was filtered and the filtrate cooled to 0° C. before methanesulfonyl chloride (3.8 mL, includes correction for residual water content) was charged. Triethylamine (21.9 mL, includes correction for residual water content) was then added over 30 min, maintaining the temperature between 0 and 7° C. The batch was held for 15 min before being heated to 18° C. over 1 h and then held at this temperature for 4 h. The reaction mixture was washed sequentially with water (2×150 mL) and sat. aq. sodium chloride (150 mL). The residual organic phase was distilled to lower the batch volume to ca. 60 mL before isopropanol (120 mL) was charged. The batch volume was further reduced to ca. 105 mL before being heated to 75° C., held for 30 min and then cooled to −5° C. over 2 h. After holding for 1 h at −5° C., the product (Compound E) was isolated by filtration and dried under reduced pressure at 43° C. (11.9 g, 83%).

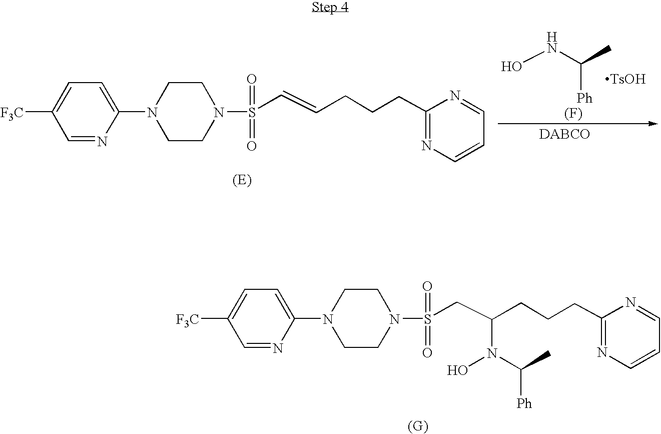

To a mixture of Compound E (25.2 g) and Compound F (26.6 g) was charged tetrahydrofuran (250 mL) and the resultant slurry stirred for 5 min at 20° C. under nitrogen. DABCO™ (13.0 g) was added in a single portion and stirring continued for 24 h at 20° C. The reaction mixture was then washed by addition of aq. disodium citrate solution (0.1 M, 200 mL) containing sodium chloride (10% w/w) and stirring for 30 min. The mixture was then allowed to settle before the lower aqueous phase was removed. The upper organic phase containing Compound G (30.3 g, 87% solution yield) was stored at <10° C. under nitrogen prior to subsequent processing.

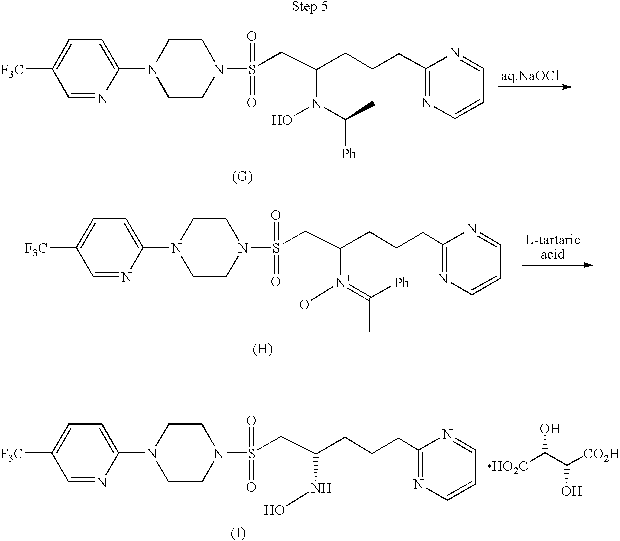

The organic phase (ca. 100 mL) from step 4 containing Compound G (13.0 g) was cooled to 15° C. with stirring under nitrogen, whereupon aq. sodium hypochlorite solution (1.3 M, 69 mL) was added slowly over 2 h whilst maintaining the temperature between 15 and 20° C. The mixture was then held at 15° C. for 1 h before the temperature was adjusted to 20° C. The mixture was allowed to settle and the lower aqueous phase was removed. The residual organic phase containing Compound H was charged to L-tartaric acid (6.7 g) suspended in isopropyl acetate (175 mL) and the reaction mixture stirred at 20° C. for 20 h, after which it was subjected to a thermal cycling procedure which consisted of heating the mixture up to 40° C. at a rate of 30° C. per hour and holding at that temperature for 30 min, then reducing the temperature to 0° C. at a rate of 10° C. per hour and holding at that temperature for 30 min. This procedure was repeated twice further, whereupon after holding at 0° C. for 3 h the product (Compound I) was isolated by filtration and dried under reduced pressure at up to 50° C. (9.4 g, 67%).

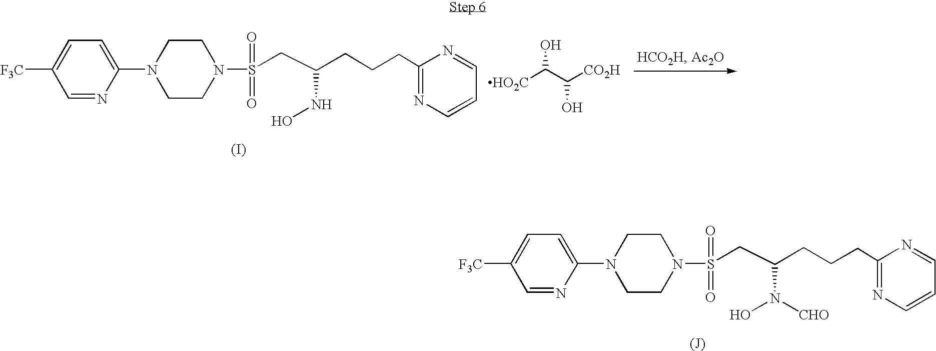

To formic acid (10 mL) was charged acetic anhydride (7.9 mL) at 0° C. under nitrogen and the mixture stirred for 1 h. This was then added slowly to Compound I (7.4 g) in formic acid (50 mL) at 0° C., maintaining the temperature between 0 and 5° C. The reaction mixture was stirred at 0° C. for 2 h. tert-Butyl methyl ether (200 mL) was then added at 0° C. followed slowly by aq. sodium hydroxide (6 M, 120 mL), whilst maintaining the temperature below 10° C. The pH of the lower aqueous phase was checked to ensure that it was at least 3.6, whereupon the phases were allowed to settle and the aqueous phase removed. Further aq. sodium hydroxide (6 M, ca. 60 mL) was added in 10 mL portions until the pH of the aqueous layer was 5.5-6.0. The mixture heated to 45° C. and held for 4 h. The phases were again allowed to settle and the lower aqueous layer was then removed. tert-Butyl methyl ether was removed by distillation from the residual organic phase until the batch volume was ca. 80 mL and ethanol (100 mL) then added. Further solvent was removed by distillation until the batch volume was ca 60 mL and the residual tert-butyl methyl ether was <10% w/w (as determined by GC analysis). The reaction mixture was cooled to 0° C. at a rate of 0.5° C. per minute and held for at least 1 h. The product (Compound 3) was isolated by filtration, washed sequentially with water (50 mL) and ethanol (20 mL), and dried under reduced pressure at 42° C. (4.46 g, 75%).

1. A process for preparing an optically active compound of formula (II) or a salt thereof 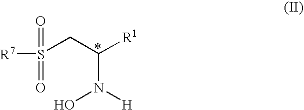
where * indicates a stereogenic centre; R¹ is an optionally substituted hydrocarbyl group; R⁷ is an optionally substituted hydrocarbyl group, or an optionally substituted heterocyclic group, which process comprises the acid hydrolysis of an optically active compound of formula (IV) 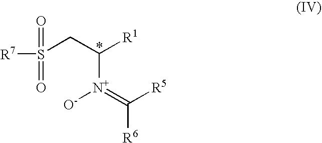
where R¹ and R⁷ are as defined above; and one of R⁵ or R⁶ is an optionally substituted aromatic group or an electron-withdrawing group and the other is an optionally substituted alkyl group, recovering the resultant optically active compound of formula (II) as a salt, and thereafter if desired, converting the salt to a compound of formula (II).

2. A process according to claim 1 wherein acid hydrolysis is carried out using an optically active acid.

3. A process according to claim 2 wherein the optically active acid is L-tartaric acid.

4. A process according to claim 1 wherein the compound of formula (IV) is prepared by reacting an optically active compound of formula (V) 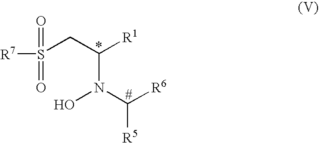
where *, R¹, R⁵, R⁶ and R⁷ are as defined in claim 1 and # represents a second stereogenic centre, with an oxidising agent.

5. A process according to claim 4 wherein the oxidising agent is an alkali metal hypochlorite.

6. A process according to claim 5 wherein the compound of formula (IV) produced is converted to a compound of formula (II) 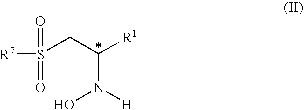
where * indicates a stereogenic centre; R¹ is an optionally substituted hydrocarbyl group; R⁷ is an optionally substituted hydrocarbyl group or an optionally substituted heterocyclic group, in situ.

7. A process according claim 4 wherein the compound of formula (V) is prepared by reacting a compound of formula (VI) 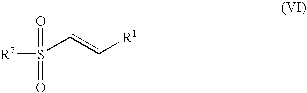
wherein R¹ is an optionally substituted hydrocarbyl group; and R⁷ is an optionally substituted hydrocarbyl group or an optionally substituted heterocyclic group,
with an optically active compound of formula (VII) or a salt thereof 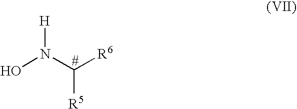
wherein R⁵ and R⁶ are as defined in claim 1 and # indicates a stereogenic centre, in the presence of a base.

8. A process according to claim 1 wherein the compound of formula (II) obtained is converted to a compound of formula (I) or a salt thereof 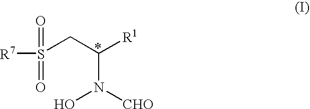
where * indicates a stereogenic centre; R¹ is an optionally substituted hydrocarbyl group; and R⁷ is an optionally substituted hydrocarbyl group, or an optionally substituted heterocyclic group,
by reaction with a compound of formula (III) 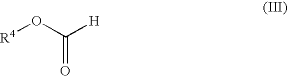
where R⁴ is an alkyl, aralkyl, aryl or acyl group, any of which may be optionally substituted.

9. A process according to claim 8 wherein R⁴ is acetyl, ethyl or 2,2,2-trifluoroethyl.

10. A process according to claim 1 wherein the compound of formula (II) is a compound of formula (IIA) 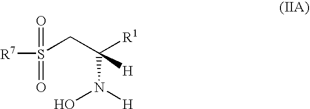
where R¹ is an optionally substituted hydrocarbyl group; and R⁷ is an optionally substituted hydrocarbyl group, or an optionally substituted heterocyclic group.

11. A process according to claim 4 wherein the compound of formula (V) is a compound of formula (VA) 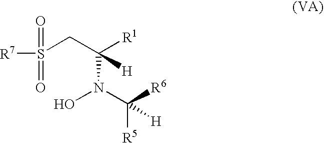
where R¹ is an optionally substituted hydrocarbyl group; R⁷ is an optionally substituted hydrocarbyl group, or an optionally substituted heterocyclic group; and one of R⁵ or R⁶ is an optionally substituted aromatic group or an electron-withdrawing group and the other is an optionally substituted alkyl group provided that R⁵ is the optionally substituted aromatic group or electron-withdrawing group, and R⁶ is alkyl.

12. A process according to claim 7 wherein the compound of formula (VII) is a compound of formula (VIIA) 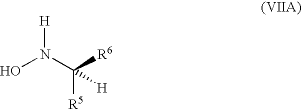
where R⁵ is an optionally substituted aromatic group or an electron-withdrawing group, and R⁶ is an alkyl group.

13. A process according to claim 8 wherein the compound of formula (II) is a compound of formula (IIA) 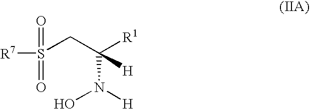
where R¹ is an optionally substituted hydrocarbyl group; and R⁷ is an optionally substituted hydrocarbyl group, or an optionally substituted heterocyclic group, and the compound of formula (I) obtained is a compound of formula (IA) or a salt thereof 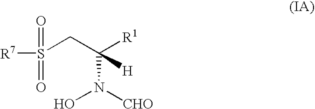
where R¹ is an optionally substituted hydrocarbyl group; and R⁷ is an optionally substituted hydrocarbyl group or an optionally substituted heterocyclic group.

14. A process according to claim 1 where wherein R¹ represents an optionally substituted group selected from C₁₋₆ alkyl, C₅₋₇ cycloalkyl, a saturated heterocyclyl, aryl, heteroaryl, aryl-C₁₋₁₆ alkyl, heteroaryl-C₁₋₆ alkyl, cycloalkyl-C₁₋₆ alkyl or saturated heterocyclyl-C₁₋₆ alkyl.

15. A process according to claim 14 wherein R¹ is substituted by one or two substituents, which may be the same or different, selected from C₁₋₄ alkyl, halogen, CF₃ and CN.

16. A process according to claim 14 wherein R¹ is 3-chlorophenyl, 4-chlorophenyl, 3-pyridyl, 2-pyridylpropyl, 2- or 4-pyrimidinylethyl (optionally monosubstituted by fluorine), 2- or 4-pyrimidinylpropyl, 2-(2-pyrimidinyl)propyl (optionally monosubstitued by fluorine) or 5-fluoro-2-pyrimidinylethyl.

17. A process according to claim 1 wherein R⁷ is a group NR²R³ where R² and R³, together with the nitrogen atom to which they are attached form an optionally substituted saturated ring, which optionally contains further heteroatoms.

18. A process according to claim 17 wherein R⁷ is a group of sub-formula (c) 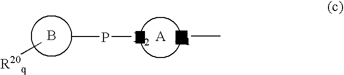
where X₁ and X₂ are independently selected from N and C; ring B is a monocyclic or bicyclic cycloalkyl, aryl or heteroaryl ring comprising up to 12 ring atoms and containing one or more heteroatoms independently chosen from N, O, and S; or ring B is may be biphenyl; or ring B may be linked to ring A by a C₁₋₄alkyl or a C₁₋₄ alkoxy chain linking the 2-position of ring B with a carbon atom a to X²; q is 0, 1, 2 or 3 and each R²⁰ is independently selected from halogen, NO₂, COOR or a group OR²³ wherein R is hydrogen or C₁₋₆ alkyl, CN, CF₃, C₁₋₆ alkyl, SC₁₋₆ alkyl, SOC₁₋₆ alkyl, SO₂C₁₋₆ alkyl, C₁₋₆ alkoxy and up to C₁₀ aryloxy and R²³ represents a group selected from C₁₋₆ alkyl or aryl, which said group is substituted by one or more fluorine groups; P is —(CH₂)ₛ— wherein s is 0, 1 or 2, or P is an alkene or alkyne chain of up to six carbon atoms; and where X₂ is C, P may be a group -Z-, —(CH[R²²])ₜ-Z-, -Z-(CH[R²²]ₜ— or -Z-(CH[R²²])ₜ-Z-, wherein Z is selected from —CO—, —S—, SO—, —SO₂—, —NR²²—, or —O— wherein t is 1 or 2, or P may be selected from —CO—N(R²²)—, —N(R²²)—CO—, —SO₂N(R²²)— and —N(R²²)SO₂—, and R²² is hydrogen, C₁₋₆ alkyl, up to C₁₀ aralkyl or up to C₉ heteroaryl; and ring A is a 5 to 7 membered saturated ring which is optionally mono- or di-substituted by groups independently selected from halogen, C₁₋₆ alkyl, C₁₋₆ alkoxy or an oxo group wherein the C₁₋₆ alkyl groups may be optionally substituted by halo.

19. A process according to claim 13 wherein the compound produced is a compound of formula (X) 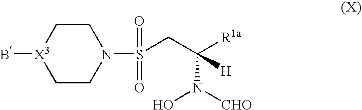
wherein B′ represents a phenyl group monosubstituted at the 3- or 4-position by halogen or trifluoromethyl, or disubstituted at the 3- and 4-positions by halogen (which may be the same or different); or B represents a 2-pyridyl or 2-pyridyloxy group monosubstituted at the 4-, 5- or 6-position by halogen, trifluoromethyl, cyano or C₁₋₄ alkyl; or B represents a 4-pyrimidinyl group optionally substituted at the 6-position by halogen or C₁₋₄ alkyl; X³ represents a carbon or nitrogen atom; R^{1a} represents a trimethyl-1-hydantoin C₂₋₄ alkyl or a trimethyl-3-hydantoin C₂₋₄ alkyl group; phenyl or C₂₋₄ alkylphenyl monosubstituted at the 3- or 4-position by halogen, trifluoromethyl, thio or C₁₋₃ alkyl or C₁₋₃ alkoxy; phenyl-SO₂NHC₂₋₄alkyl; 2-pyridyl or 2-pyridyl C₂₋₄ alkyl; 3-pyridyl or 3-pyridyl C₂₋₄ alkyl; 2-pyrimidine-SCH₂CH₂; 2- or 4-pyrimidinyl C₂₋₄ alkyl optionally monosubstituted by one of halogen, trifluoromethyl, C₁₋₃ alkyl, C₁₋₃ alkyloxy, 2-pyrazinyl optionally substituted by halogen or 2-pyrazinyl C₂₋₄ alkyl optionally substituted by halogen.

20. A process according to claim 13 wherein the compound obtained is a compound of formula (XI) or a pharmaceutically acceptable salt, prodrug or solvate thereof 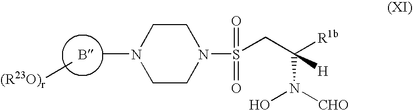
wherein ring B″ represents a monocyclic aryl ring having six ring atoms or a monocyclic heteroaryl ring having up to six ring atoms and containing one or more ring heteroatoms wherein each said heteroatom is nitrogen;
R²³ is as defined above; r is 1, 2 or 3; and R^{1b} represents an optionally substituted group selected from C₁₋₆ alkyl, C₅₋₇ cycloalkyl, a saturated heterocyclyl, aryl, heteroaryl, aryl-C₁₋₁₆ alkyl, heteroaryl-C₁₋₆ alkyl, cycloalkyl-C₁₋₆ alkyl or saturated heterocyclyl-C₁₋₆ alkyl.

21. A compound of formula (IV) as defined in claim 1.

22. A salt of a compound of formula (IIA) as defined in claim 10 and an optically active acid.

23. A compound of formula (V) as defined in claim 4.

24. A compound of formula (VA) as defined in claim 11, where R⁷ is a group NR²R³ where R² and R³, together with the nitrogen atom to which they are attached form an optionally substituted saturated ring, which optionally contains further heteroatoms.
